(19) United States
(12) Patent Application Publication
(10) Pub. No.: US 2004/0006072 A1
(43) Pub. Date: Jan. 8, 2004

(54) Sustained-release alprazolam composition

(76) Inventors: Robert M. Franz, Raleigh, NC (US); Leo Lucisano, Cary, NC (US); Phillip R. Nixon, Portage, MI (US); Mark G. Eller, Kansas City, MO (US); Joseph C. Fleishaker, Kalamazoo, MI (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/464,045

(22) Filed: Jun. 18, 2003

Related U.S. Application Data
(63) Continuation-in-part of application No. 10/179,706, filed on Jun. 25, 2002, now abandoned.

(51) Int. Cl. A61K 31/551; A61K 31/716

(52) U.S. Cl. 514/220; 514/57

(57) ABSTRACT

A sustained-release pharmaceutical composition in a form of a tablet comprises (a) alprazolam in an amount of about 0.1 mg to about 5 mg per tablet, (b) a high viscosity HPMC and (c) a low viscosity HPMC, wherein the total amount of HPMC is about 110 mg to about 135 mg per tablet and the high and low viscosity HPMCs are present in a weight ratio of about 40:60 to about 60:40. The tablet can be a member of a series having different amounts of alprazolam in the range from about 0.1 mg to about 5 mg, members of the series having substantially equal total tablet weight and being substantially bioequivalent. The composition is useful in treatment of CNS conditions and disorders including general anxiety disorder, anxiety associated with depression, panic disorder and panic attacks.

This application is a continuation-in-part of application Ser. No. 10/179,706, filed on Jun. 25, 2002.

### FIELD OF THE INVENTION

The present invention relates to a pharmaceutical composition useful in treatment of disorders of the central nervous system (CNS), particularly disorders wherein a benzodiazepine therapeutic agent is indicated, for example anxiety and panic disorder. More specifically, it relates to a sustained-release alprazolam composition.

### BACKGROUND OF THE INVENTION

Alprazolam, a member of the 1,4-benzodiazepine class of CNS-active compounds, is an effective anxiolytic and anti-panic agent. The immediate-release alprazolam tablet formulation currently marketed as Xanax® tablets by Pharmacia Corporation can be prescribed for administration of up to four doses per day for treatment of anxiety and, in some instances, in excess of four doses per day for treatment of panic disorder. Such frequency of dosing is inconvenient and can adversely affect patient compliance. In addition, breakthrough anxiety can be a problem with current dosing regimens. Therefore, an extended- or sustained-release formulation of alprazolam would be advantageous to reduce frequency of dosing, and provide more uniform blood levels of the drug for continuous control of symptoms, greater ease of discontinuation and greater patient safety.

Sustained-release alprazolam formulations have been investigated, including formulations wherein alprazolam is dispersed in a polymer matrix, for example a hydroxypropylmethylcellulose (HPMC) matrix. Franz et al. (1987), Journal of Controlled Release 5, 159-172, examined effects of several formulation variables on in vitro alprazolam release rate from such a matrix formulation comprising HPMCs of different viscosity grades, sodium carboxymethylcellulose (sodium CMC) and lactose. These variables included ratio of high to low viscosity HPMC, ratio of sodium CMC to lactose, and matrix drug loading. Franz et al. established relationships among these variables, but failed to provide guidance on absolute amounts of HPMC to be formulated with desired amounts of alprazolam.

Attempts have also been made to develop a mathematical model to predict relative drug release rate as a function of formulation composition for HPMC-based extended-release alprazolam tablets. Gao et al. (1995), Pharmaceutical Research 12(7), 965-971, found that the predictive power of their model became poor as drug load increased. Again, no teaching was provided on absolute amount of HPMC.

Busto et al (2000), Journal of Clinical Psychopharmacology 20(6), 628-635, have published pharmacokinetic (PK) data derived from single-dose (1.5 mg) administration of an unspecified sustained-release capsule formulation of alprazolam. An essentially constant plasma concentration of alprazolam was observed over a 6-14 hour period.

Mumford et al. (1995), Clinical Pharmacology & Therapeutics 57(3), 356-365, presented PK data derived from single-dose (2 mg and 3 mg) administration of an unspecified extended-release formulation of alprazolam.

HPMC as a sustained-release matrix polymer is well known in the art, but amounts of HPMC giving suitable in vitro, much less in vivo, drug release properties for a range of drug loadings are not generally predictable.

Xanax® tablets, an immediate-release formulation of alprazolam, are designed to be divided by the patient into equally sized portions for dose control. See Physicians' Desk Reference, 56th edition (2002), pp. 2865-2869. Dividing by the patient is generally not appropriate for extended-release dosage forms which typically are designed to be administered whole. A problem in the case of an alprazolam extended-release dosage form is that a wide range of dosage amounts is prescribable, depending on the nature and severity of the disorder being treated; furthermore certain treatment regimens require different dosage amounts to be administered on different days. Franz et al., op. cit., disclosed that over a range of alprazolam dosage strengths from 1.05 to 3.15 mg, in vitro release rate of the drug from HPMC matrix tablets varied considerably.

It would be of great benefit to provide a sustained-release alprazolam formulation system that is adaptable to a broad range of dosage strengths, yet exhibits substantial bioequivalence when different dosage strengths are administered in an equal total dosage amount. For example, it would be advantageous if two 0.5 mg dosage forms taken together were bioequivalent to one 1 mg dosage form, or if three 1 mg dosage forms taken together were bioequivalent to one 3 mg dosage form. One approach that might achieve such a benefit would be to provide all dosage strengths in an identical formulation, wherein higher dosage strengths are accommodated in proportionally larger dosage units. Thus, according to such an approach, a 3 mg tablet would have a total weight six times greater than a 0.5 mg tablet.

However, it is found preferable to provide all dosage strengths in tablets of similar total weight. Advantages include avoidance of excessively large tablets for the higher dosage strengths, which can be difficult to swallow and result in poor patient compliance; and avoidance of excessively small tablets for the lower dosage strengths, which can be difficult to handle by patients lacking manual dexterity, for example geriatric or arthritic patients, or having impaired vision. There are also benefits in manufacturing and packaging costs where tablet size is more uniform.

Thus a sustained-release formulation system for alprazolam is desired that accommodates dosage strengths over at least a six-fold range from 0.5 mg to 3 mg in substantially the same total dosage form weight, yet exhibits bioequivalence over a corresponding range of excipient/drug weight ratios. The in vitro data of Franz et al., op. cit., are not promising in this regard, in that tablets of higher dosage strength, having a low excipient/drug weight ratio, released alprazolam more slowly than tablets of lower dosage strength, at least in absence of sodium CMC.

A key to solving this problem would be to identify, for a sustained-release dosage form utilizing an HPMC matrix, a suitable amount of HPMC giving substantially equivalent in vivo release and absorption rates over a broad range of alprazolam loadings. No suggestion that such an amount is practically attainable, or what such an amount might be, is found in the cited Franz et al. disclosure.

### SUMMARY OF THE INVENTION

It is surprisingly found that the objectives of this invention are met by a sustained-release alprazolam tablet comprising a particular blend of high and low viscosity HPMCs in a total amount of about 110 mg to about 135 mg per tablet. Accordingly, there is now provided a pharmaceutical composition in a form of a tablet comprising (a) alprazolam in an amount of about 0.1 mg to about 5 mg per tablet, (b) a high viscosity HPMC and (c) a low viscosity HPMC, wherein the total amount of HPMC is about 110 mg to about 135 mg per tablet and the high and low viscosity HPMCs are present in a weight ratio of about 40:60 to about 60:40.

In a particular embodiment, the tablet is a member of a series having different amounts of alprazolam in the range from about 0.1 mg to about 5 mg, members of the series having substantially equal total tablet weight.

There is further provided a method of treating a subject having a CNS disorder, more particularly a disorder wherein a benzodiazepine therapeutic agent is indicated, comprising a step of orally administering to the subject a therapeutically effective amount of a pharmaceutical composition as provided herein.

### BRIEF DESCRIPTION OF THE DRAWINGS

FIG. 1 shows in vitro drug release profiles for 0.5 mg, 1 mg, 2 mg and 3 mg sustained-release alprazolam tablets of Examples 1-4 respectively.

FIG. 2 shows mean alprazolam plasma concentration profiles in human subjects administered a total of 6 mg alprazolam in the form of 1, 2 and 3 mg sustained-release alprazolam tablets of Examples 2-4 respectively.

FIG. 3 shows mean alprazolam plasma concentration profiles in human subjects administered a total of 1 mg alprazolam in the form of 0.5 and 1 mg sustained-release alprazolam tablets of Examples 1 and 2 respectively.

### DETAILED DESCRIPTION OF THE INVENTION

Illustrative methods for preparation of alprazolam, 8-chloro-1-methyl-6-phenyl-4H-s-triazolo-[4,3-α]-1,4-benzodiazepine (I), are disclosed in the patents individually listed below and incorporated herein by reference.

U.S. Pat. No. 3,709,898 to Hester.

U.S. Pat. No. 3,879,413 to Hester.

U.S. Pat. No. 3,980,789 to Hester.

U.S. Pat. No. 3,987,052 to Hester.

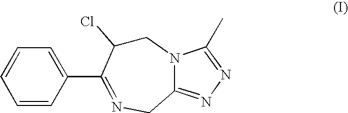

Any pharmaceutically acceptable form of alprazolam can be used, including any suitable crystalline or other solid state form, enantiomer or tautomer thereof.

In a tablet of the invention, alprazolam is present in an amount of about 0.1 mg to about 5 mg, preferably about 0.5 to about 3 mg, for example about 0.5 mg, about 1 mg, about 2 mg or about 3 mg.

The alprazolam is distributed in a matrix that comprises HPMC, optionally but preferably together with other excipients as detailed below. HPMC is believed to function as a release-controlling agent and a binder in the formulation of the invention. The HPMC is present in a total amount of about 110 mg to about 135 mg, for example about 120 mg, per tablet. It has surprisingly been found that in such an amount, the HPMC provides in vivo release, as determined by PK data, that is substantially unaffected by alprazolam loading in the range provided above, even where in vitro release data would predict a significant effect of alprazolam loading on in vivo release rates.

HPMC is commercially available in various grades, under several trade names, including Methocel® E, F, J and K (all previously designated as Methocel® HG) of Dow Chemical Co., U.S.A., HPM of British Celanese Ltd., U.K., and Metalose® SH of Shin-Etsu Ltd., Japan. The various grades available under a given trade name typically represent differences in methoxy and hydroxypropoxy content as well as molecular weight of the HPMC.

A preferred type is HPMC 2208, which contains about 19% to about 24% by weight of methoxy substituents, and about 4% to about 12% by weight of hydroxypropoxy substituents, calculated on a dry basis.

Viscosity of commercial HPMCs ranges from about 2 to about 225,000 cP (centipoise), as measured in a 2% aqueous solution at 20° C. The term “high viscosity HPMC” herein refers to HPMC having a viscosity of about 1,500 to about 225,000 cP, and the term “low viscosity HPMC” herein refers to HPMC having a viscosity of about 2 to about 400 cP.

A preferred high viscosity HPMC is HPMC 2208 having a viscosity of about 3000 to about 5600 cP, which is illustratively available as Methocel® K4 MP of Dow. A preferred low viscosity HPMC is HPMC 2208 having a viscosity of about 80 to about 120 cP, which is illustratively available as Methocel® K100LVP of Dow. Equivalent products are available from other manufacturers.

Both a high viscosity and a low viscosity HPMC are present in the composition. In one embodiment, both high and low viscosity HPMCs conform to the preferred types described above. The weight ratio of high to low viscosity HPMC is about 40:60 to about 60:40, preferably about 45:55 to about 55:45, for example about 1:1. Each of the high and low viscosity HPMCs can be present in an amount of about 50 mg to about 70 mg per tablet, for example about 60 mg per tablet.

Preferably the tablet comprises one or more additional pharmaceutically acceptable excipients other than the high and low viscosity HPMCs. Such excipients include conventional pharmaceutical tablet excipients, for example diluents, binders, disintegrants, glidants, lubricants, pH modifying agents, coloring agents, antioxidants, etc.

In one embodiment, a diluent is present. A preferred diluent is lactose. Either lactose monohydrate or anhydrous lactose can be used. A suitable amount of lactose is about 150 mg to about 300 mg, preferably about 180 mg to about 260 mg, more preferably about 200 mg to about 240 mg per tablet.

In another embodiment, a disintegrant is present. For example, sodium CMC (carmellose sodium) can be used as a disintegrant in a composition of the invention, but preferably the amount of sodium CMC is zero to about 50 mg per tablet. More preferably substantially no sodium CMC is present.

In yet another embodiment, a glidant is present. A preferred glidant is colloidal silicon dioxide, suitably ii an amount of about 0.3 mg to about 1.5 mg, preferably about 0.6 mg to about 0.9 mg, per tablet.

In yet another embodiment, a lubricant is present. A preferred lubricant is magnesium stearate, suitably in an amount of about 1 mg to about 2 mg, preferably about 1.3 mg to about 1.7 mg, per tablet.

Optionally, one or more coloring agents can be present in the composition. Selection of coloring agents can be made, for example, so that tablets of different dosage strengths can be easily distinguished. Illustratively, D&C Yellow #10 can be present in an amount of about 0.2 mg to about 0.3 mg per tablet, and/or FD&C Blue #2 can be present in an amount of about 0.05 mg to about 0.09 mg per tablet. In one embodiment, D&C Yellow #10 and FD&C Blue #2 are used in combination as a coloring agent.

The phrase “pharmaceutically acceptable” is employed herein to refer to compounds, materials, compositions and/or dosage forms that are, within the scope of sound medical judgment, suitable for use in contact with tissues of human beings and animals and without excessive toxicity, irritation, allergic response, or any other problem or complication, commensurate with a reasonable benefit/risk ratio.

Amounts of excipient ingredients specified herein are consistent with a tablet size that is neither inconveniently small nor so large as to present difficulty in swallowing by most subjects, it being noted that to obtain the full benefit of the sustained-release properties of a tablet of the invention it should be swallowed whole. Typically, total tablet weight is about 200 mg to about 500 mg, preferably about 250 mg to about 450 mg, more preferably about 300 mg to about 400 mg, for example about 350 mg.

In a preferred embodiment, the tablet is a member of a series having different amounts of alprazolam in the range from about 0.1 mg to about 5 mg, members of the series having substantially equal total tablet weight. For example, tablets in the series can have amounts of alprazolam of about 0.5 mg, about 1 mg, about 2 mg and about 3 mg per tablet respectively.

Surprisingly, members of such a series, when formulated according to the invention, are substantially bioequivalent. The term “substantially bioequivalent” herein means that a first composition in the series exhibits a mean value of the important PK parameters Cₘₐₓ (maximum plasma concentration of alprazolam) and/or AUC (area under the plasma concentration/time curve, a measure of overall bioavailability) that is about 80% to about 125% of the corresponding mean value exhibited by a second composition in the series, in a standard PK study in adult humans wherein equal doses of the two compositions are administered. Preferably members of the series exhibit substantially similar mean plasma concentration/time curves in a standard PK study in adult humans.

The invention also provides a method of treating a CNS condition or disorder in a subject. The method comprises orally administering to the subject a therapeutically effective amount of a sustained-release pharmaceutical composition as provided herein. The composition provides an alprazolam release rate that is acceptable for once or twice daily dosing in humans, thus in a preferred method a composition of the invention is orally administered in a therapeutically effective amount to a human subject one or two times per day.

CNS conditions and disorders include those having a neurologic and/or a psychiatric component. Illustrative CNS conditions and disorders include, for example, personality disorders including paranoid, schizoid, schizotypal, bipolar, histrionic, delusional, narcissistic, emotionally unstable, psychopathic and sociopathic personality disorders; habit and impulse disorders including pathological gambling, stealing, trichotillomania, etc.; obsessive-compulsive disorder; passive-aggressive disorder; acute and transient psychotic disorders; psychotic depression; schizoaffective disorder; hypochondria; cyclothymia; dysthymia; manic-depressive illness; major depressive disorder; treatment-resistant depression; adult and childhood onset schizophrenias; drug dependence including harmful use and abuse of, addiction to or dependence on opioids, narcotics, barbiturates, alcohol, benzodiazepines, amphetamines, cocaine, cannabinoids, hallucinogens, stimulants, nicotine (tobacco) and solvents; withdrawal states and mood and psychotic disorders related to such dependence; sexual dysfunction including hypoactive sexual desire disorder, sexual aversion or avoidance and erectile dysfunction; gender identity disorders; sexual preference disorders; general anxiety disorder; social anxiety disorder; mixed anxiety and depressive disorder; attention deficit hyperactivity disorder (ADHD) and depression and anxiety associated therewith; depression, anxiety, emotional dysregulation and behavioral disturbances associated with mental retardation; developmental disorders including autism, Asperger's syndrome and Rett's syndrome; childhood conduct and attachment disorders; premenstrual dysphoric disorder; postpartum depression; phobias including social phobias, agoraphobia and specific phobias related for example to hospitals, injections, venesection, etc.; posttraumatic stress disorder; dissociative disorder; Briquet's syndrome; affective disorders including depression, bipolar affective disorder and recurrent depressive disorder; organic mood, anxiety and emotionally labile disorders resulting for example from brain damage or dysfunction arising from head injury, intracranial masses, stroke, etc.; chronic fatigue; stress-induced psychotic episodes; dementia including presenile dementia, Pick's disease, vascular dementia, multi-infarct dementia, Alzheimer's disease, dementia associated with Creutzfeldt-Jakob disease and HIV-related dementia; other neurodegenerative disorders including Parkinson's disease and Huntington's disease; suicidal behavior; eating disorders including anorexia, bulimia and binge eating disorder; adjustment disorders; somatization disorder; somatoform autonomic dysfunction; somatoform pain disorder; panic attacks; panic disorder; amnesia; neuropathic pain; fibromyalgia; migraine; epilepsy; tinnitus; enuresis; sleep disorders including insomnia, hypersomnia, narcolepsy, nightmares and night terrors; delirium; postconcussion syndrome; multiple sclerosis; tremors; muscular spasms; restless legs syndrome; Lennox-Gastaut syndrome; motor and vocal tic disorders; Tourette's syndrome; supranuclear palsy; Shy-Drager syndrome; trigeminal neuralgia; Bell's palsy; motor neuron diseases such as amyotrophic lateral sclerosis; and psychosomatic and psychosocial conditions associated with non-CNS disorders such as diabetes, inflammatory disease, infertility, allergies, psoriasis, asthma, hypertension, overactive bladder, thyroid disorders, obesity, immune disorders and cancer.

More specifically, compositions of the invention can be used to treat any condition or disorder that is responsive to benzodiazepine drugs, and are especially useful in treatment or management of general anxiety disorder, anxiety associated with depression, panic disorder and panic attacks.

The sustained-release alprazolam composition of the present invention can be packaged in a container, accompanied for example by a package insert providing pertinent information such as dosage and administration information, contraindications, precautions, drug interactions and adverse reactions.

### EXAMPLES

### Example 1

Tablets having the composition shown in Table 1 were prepared in a lot of 187,500 tablets. These were nominally 0.5 mg alprazolam tablets, but with a calculated alprazolam content of 0.525 mg per tablet.

TABLE 1
Composition of 0.5 mg alprazolam sustained-release tablets

| | Amount | Weight | Weight/tablet |
|---|---|---|---|
| Component | (kg) | (%) | (mg) |
| alprazolam | 0.0984 | 0.15 | 0.525 |
| lactose | 41.57 | 64.14 | 221.7 |
| HPMC type 2208 USP, 4000 cP | 11.36 | 17.53 | 60.6 |
| HPMC type 2208 USP, 100 cP | 11.36 | 17.53 | 60.6 |
| colloidal silicon dioxide | 0.1406 | 0.22 | 0.75 |
| magnesium stearate | 0.2813 | 0.44 | 1.5 |
| total | 64.81 | 100 | 345.7 |

The alprazolam and all excipients except the magnesium stearate were passed through a screen using a Quick Sieve equipped with a 0.8 mm sieve drum and stator #1 and charged into a 40 cu. ft. Patterson-Kelley V-Blender, where they were mixed together for approximately 20 minutes until uniformly blended. If a uniform blend was not achieved, the mixture was passed through a screen and mixed together again in the blender. Next, the magnesium stearate was passed through a #20 mesh screen with 3-5 kg of the blended alprazolam material. The resulting magnesium stearate mixture was charged into the V-Blender containing the balance of the blended alprazolam material, and mixed for approximately 3 minutes. The resulting tableting mixture was compressed into tablets using a Manesty Mark IV rotary tablet press.

### Example 2

Tablets having the composition shown in Table 2 were prepared in a lot of 187,500 tablets, by the process described in Example 1. These were nominally 1 mg alprazolam tablets, but with a calculated alprazolam content of 1.05 mg per tablet.

TABLE 2
Composition of 1 mg alprazolam sustained-release tablets

| | Amount | Weight | Weight/tablet |
|---|---|---|---|
| Component | (kg) | (%) | (mg) |
| alprazolam | 0.1969 | 0.30 | 1.05 |
| lactose | 41.57 | 64.00 | 221.7 |
| HPMC type 2208 USP, 4000 cP | 11.36 | 17.49 | 60.6 |
| HPMC type 2208 USP, 100 cP | 11.36 | 17.49 | 60.6 |
| colloidal silicon dioxide | 0.1406 | 0.22 | 0.75 |
| magnesium stearate | 0.2813 | 0.43 | 1.5 |
| D&C Yellow #10 | 0.045 | 0.07 | 0.24 |
| total | 64.95 | 100 | 346.4 |

### Example 3

Tablets having the composition shown in Table 3 were prepared in a lot of 187,500 tablets, by the process described in Example 1. These were nominally 2 mg alprazolam tablets, but with a calculated alprazolam content of 2.1 mg per tablet.

TABLE 3
Composition of 2 mg alprazolam sustained-release tablets

| | Amount | Weight | Weight/tablet |
|---|---|---|---|
| Component | (kg) | (%) | (mg) |
| alprazolam | 0.3938 | 0.60 | 2.1 |
| lactose | 41.57 | 63.84 | 221.7 |
| HPMC type 2208 USP, 4000 cP | 11.36 | 17.44 | 60.6 |
| HPMC type 2208 USP, 100 cP | 11.36 | 17.44 | 60.6 |
| colloidal silicon dioxide | 0.1406 | 0.22 | 0.75 |
| magnesium stearate | 0.2813 | 0.43 | 1.5 |
| FD&C Blue #2 Aluminum Lake | 0.015 | 0.02 | 0.08 |
| total | 65.12 | 100 | 347.3 |

### Example 4

Tablets having the composition shown in Table 4 were prepared in a lot of 187,500 tablets, by the process described in Example 1. These were nominally 3 mg alprazolam tablets, but with a calculated alprazolam content of 3.15 mg per tablet.

TABLE 4
Composition of 3 mg alprazolam sustained-release tablets

| | Amount | Weight | Weight/tablet |
|---|---|---|---|
| Component | (kg) | (%) | (mg) |
| alprazolam | 0.5906 | 0.90 | 3.15 |
| lactose | 41.57 | 63.60 | 221.7 |
| HPMC type 2208 USP, 4000 cP | 11.36 | 17.38 | 60.6 |
| HPMC type 2208 USP, 100 cP | 11.36 | 17.38 | 60.6 |
| colloidal silicon dioxide | 0.1406 | 0.22 | 0.75 |
| magnesium stearate | 0.2813 | 0.43 | 1.5 |
| D&C Yellow #10 | 0.045 | 0.07 | 0.24 |
| FD&C Blue #2 Alummum Lake | 0.0113 | 0.02 | 0.06 |
| total | 65.36 | 100 | 348.6 |

### Example 5

In vitro drug release rates were determined for the tablets of Examples 1-4 using USP apparatus 1 (rotating basket) at 100 rpm and 500 ml of 0.07M phosphate buffer at pH 6 as dissolution medium. Samples of the medium were removed at 1, 2, 4, 8, 12, 16 and 20 hours after immersion. Alprazolam concentrations were determined by HPLC using conventional UV absorbance detectors at 254 nm.

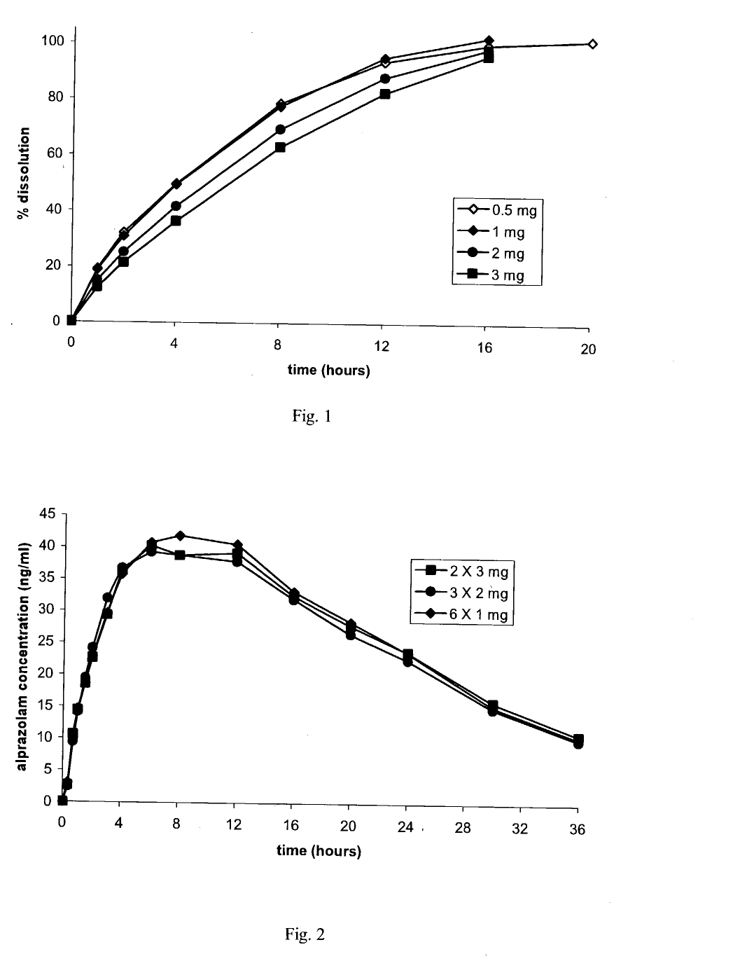

Data are shown in FIG. 1. It will be noticed that, consistent with data published by Franz et al., op. cit., as alprazolam loading in the tablet increased, release rate significantly decreased. That is, increasing the amount of alprazolam in the matrix while maintaining constant HPMC content decreased the percentage release rate of alprazolam from the matrix. These results are not suggestive of a formulation system that meets a major objective of the present invention, namely to provide bioequivalence over a wide range of alprazolam loadings.

### Example 6

Bioequivalence of the 1 mg, 2 mg and 3 mg sustained-release alprazolam tablets of Examples 2-4 was evaluated by measuring mean alprazolam plasma concentration in human subjects over a predetermined period of time following oral administration of equal 6 mg doses in a PK study. The study involved 24 healthy male volunteers, as determined by physical examination and standard clinical laboratory tests, who received each of three treatments listed below as single oral doses according to a three-way crossover design with a seven day washout period between phases.

The first treatment was administration of two 3 mg alprazolam tablets of Example 4, the second treatment was administration of three 2 mg alprazolam tablets of Example 3 and the third treatment was administration of six 1 mg alprazolam tablets of Example 2.

After receiving a cupcake and a caffeine-free beverage, subjects were required to fast from 10 p.m. the night before dosing until 4 hours after drug administration. During the fasting period no food or beverage other than water were consumed. Treatments were administered at 7 a.m., with 180 ml of water. Standard meals were consumed at 11 a.m. and 5 p.m. on the day of dosing. Subjects were allowed to remain sedentary during the study period.

Venous blood samples were collected immediately prior to drug administration and at 20 minutes, 40 minutes, 1, 1.5, 2, 3, 4, 6, 8, 12, 16, 20, 24, 30 and 36 hours after drug administration. Blood samples (10 ml) were collected into heparinized vacutainers at each sampling time. Plasma was harvested from the samples after centrifugation and frozen at −20° C. until analyzed. Determinations of alprazolam in plasma were performed by HPLC. The analytical method involved liquid-solid extraction of alprazolam and triazolam (internal standard) on an end-capped cyano-column with acetonitrile. The samples were chromatographed under isocratic conditions on a silica column using a sensitive ultraviolet detector for quantitation.

Mean plasma concentrations of alprazolam are shown in FIG. 2.

Effects of treatment on PK parameters among the three treatments were assessed by analysis of variance (ANOVA), with group, treatment and period as fixed effects and subject within group as a random effect. Differences between treatments were determined by Waller-Duncan K-ratio test and least squares means analysis. Statistical analysis was performed using SAS. The bioequivalence (on a potency-corrected basis) of the 1 mg, 2 mg and 3 mg sustained release alprazolam tablets was also assessed by 90% confidence interval analysis (two-one sided t-tests).

No significant differences were observed in plasma alprazolam concentrations between any of the treatments at any sampling time, leading to the conclusion that the 1 mg, 2 mg and 3 mg sustained release alprazolam tablet dosage formulations are bioequivalent.

### Example 7

Bioequivalence of the 0.5 mg and 1 mg sustained-release alprazolam tablets of Examples 1 and 2 respectively was evaluated in a PK study conducted according to a similar protocol to that of Example 6. Results are shown in Table 3.

The 0.5 mg and 1 mg alprazolam tablets were found to be bioequivalent. By reference to Example 6, it can be concluded that the 0.5 mg tablet is also bioequivalent to the 2 mg and 3 mg tablets because all are bioequivalent to the 1 mg tablet.

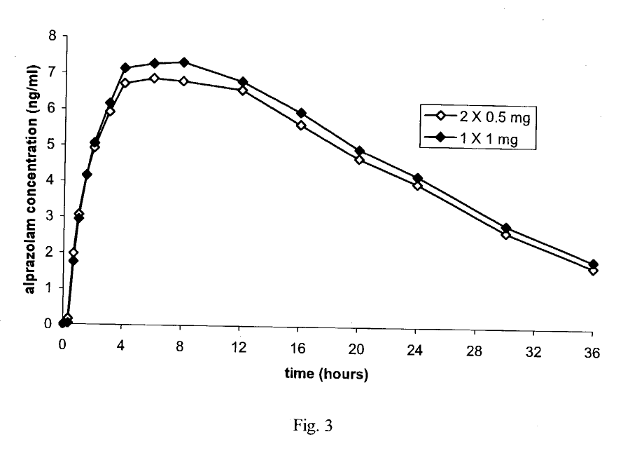

It is particularly surprising, in view of the unpromising in vitro data of Example 5, as shown in FIG. 1, that in vivo release and absorption of alprazolam, as illustrated in Examples 6 (FIG. 2) and 7 (FIG. 3), are substantially unaffected by alprazolam loading, at least where the amount of HPMC in the matrix is as provided herein.

What is claimed is:

1. A sustained-release pharmaceutical composition in a form of a tablet comprising (a) alprazolam in an amount of about 0.1 mg to about 5 mg per tablet, (b) high viscosity HPMC and (c) low viscosity HPMC, wherein the total amount of HPMC is about 110 mg to about 135 mg per tablet and the high and low viscosity HPMCs are present in a weight ratio of about 40:60 to about 60:40.

2. The composition of claim 1 that is a member of a series having different amounts of alprazolam in the range from about 0.1 mg to about 5 mg per tablet, wherein members of the series have substantially equal total tablet weight.

3. The composition of claim 2 wherein members of the series are substantially bioequivalent.

4. The composition of claim 1 wherein the alprazolam is present in an amount of about 0.5 mg to about 3 mg per tablet.

5. The composition of claim 4 that is a member of a series having respectively about 0.5 mg, about 1 mg, about 2 mg and about 3 mg alprazolam per tablet, wherein members of the series have substantially equal total tablet weight.

6. The composition of claim 5 wherein members of the series are substantially bioequivalent.

7. The composition of claim 1 having a total tablet weight of about 200 mg to about 500 mg.

8. The composition of claim 1 having a total tablet weight of about 300 mg to about 400 mg.

9. The composition of claim 1 wherein at least one of the high viscosity HPMC and the low viscosity HPMC is of type 2208.

10. The composition of claim 1 wherein the high viscosity HPMC has a viscosity of about 3000 to about 5600 cP.

11. The composition of claim 10 wherein the high viscosity HPMC is of type 2208.

12. The composition of claim 1 wherein the low viscosity HPMC has a viscosity of about 80 to about 120 cP.

13. The composition of claim 12 wherein the low viscosity HPMC is of type 2208.

14. The composition of claim 1 wherein the high viscosity HPMC is of type 2208 and has a viscosity of about 3000 to about 5600 cP and wherein the low viscosity HPMC is of type 2208 and has a viscosity of about 80 to about 120 cP.

15. The composition of claim 14 wherein the weight ratio of the high viscosity HPMC to the low viscosity HPMC is about 1:1.

16. The composition of claim 1 wherein the weight ratio of the high viscosity HPMC to the low viscosity HPMC is about 1:1.

17. The composition of claim 1, further comprising lactose in an amount of about 150 mg to about 300 mg per tablet.

18. The composition of claim 17 wherein the lactose is present in an amount of about 200 mg to about 240 mg per tablet.

19. The composition of claim 1 that contains substantially no sodium CMC.

20. A sustained-release pharmaceutical composition in a form of a tablet comprising (a) alprazolam in an amount of about 0.5 mg, about 1 mg, about 2 mg or about 3 mg per tablet; (b) HPMC type 2208 having a viscosity of about 3000 to about 5600 cP in an amount of about 50 mg to about 70 mg per tablet; (c) HPMC type 2208 having a viscosity of about 80 to about 120 cP in an amount of about 50 mg to about 70 mg per tablet; and (d) lactose in an amount of about 150 mg to about 300 mg per tablet.

21. A method of treating a CNS condition or disorder in a subject, the method comprising orally administering to the subject a therapeutically effective amount of the composition of claim 1.

22. The method of claim 21 wherein the composition is orally administered one or two times per day.

23. The method of claim 21 wherein the CNS condition or disorder is selected from the group consisting of general anxiety disorder, anxiety associated with depression, panic disorder and panic attacks.
